(19) United States
(12) Patent Application Publication
(10) Pub. No.: US 2017/0016901 A1
(43) Pub. Date: Jan. 19, 2017

(54) EXAMINATION METHOD OF BREAST CANCER

(71) Applicants: SHIZUOKA PREFECTURAL UNIVERSITY CORPORATION, Shizuoka-shi (JP); TECHNOSURUGA LABORATORY CO., LTD., Shizuoka-shi (JP); KENDAIGAKARI CO., LTD., Shizuoka-shi (JP)

(72) Inventors: Toshimasa TOYO'OKA, Shizuoka-shi (JP); Takahiro TAKAYAMA, Fuji-shi (JP)

(73) Assignees: SHIZUOKA PREFECTURAL UNIVERSITY CORPORATION, Shizuoka-shi (JP); TECHNOSURUGA LABORATORY CO., LTD., Shizuoka-shi (JP); KENDAIGAKARI CO., LTD., Shizuoka-shi (JP)

(21) Appl. No.: 15/209,005

(22) Filed: Jul. 13, 2016

(30) Foreign Application Priority Data
Jul. 14, 2015 (JP) 2015-140888

(51) Int. Cl. G01N 33/574

(52) U.S. Cl. CPC G01N 33/57415; G01N 2560/00

(57) ABSTRACT

It is an object to provide a simplified early diagnosis method for breast cancer, wherein a sample collected from a human subject is analyzed and determination of the presence or absence of breast cancer is made from the proportion of plural types of polyamines including N1-acetylspermidine.

### TECHNICAL FIELD

The present invention relates to an examination method of breast cancer using a biological sample.

### BACKGROUND ART

Breast cancer is a disease of which the incidence rate has been increased year by year. As examination method of breast cancer, palpation, MRI, mammography and biopsy are generally used. These methods all need burdens on patients, and under the present circumstances, breast cancer is found when symptom is commonly observed with advance of stage. It is therefore demanded to develop more simplified early diagnosis methods.

On the other hand, it has been reported that several polyamines are increased in blood serum, urine and saliva of cancer patients (Non-patent Documents 1 and 2).

### PRIOR ART DOCUMENTS

### Non-Patent Documents

Non-patent Document 1: J. Cancer Res. Clin. Oncol. 1997, 123, 539-545
Non-patent Document 2: Anal. Chem. 2013, 85, 11835-11842

### SUMMARY OF INVENTION

### Problems that the Invention is to Solve

The problem that the invention is to solve is to provide a simplified early diagnosis method of breast cancer.

### Means of Solving the Problems

In order to solve the above problems, the present inventors have paid attention to saliva as a sample that can easily be collected and does not bring burdens on patients. They have conducted exhaustive analysis of metabolite, specifically by metabolome analysis using saliva of breast cancer patients to conduct marker search, and found a tendency that almost all polyamines are increased in patients' group, and it was revealed that especially spermine and spermidine type metabolites showed significant difference in t-test.

However, as the results of detailed studies, individual difference is in total amount was substantially large, and it was considered that erroneous determination would be caused if a cutoff value is set with an absolute concentration. Accordingly, the differences of patterns by proportion were compared so as to minimize the influence of individual difference, and it was found that the breast cancer can be determined with high precision by using the proportion of plural types of polyamines including N1-acetylspermidine. On the basis of this finding, they have accomplished the present invention.

The present invention is summarized below.

(1) An examination method of breast cancer which comprises analyzing a sample collected from a human subject and determining the presence or absence of breast cancer by a proportion of plural types of polyamines which are selected from a polyamine and a mono- or di-acetyl form thereof and include N1-acetylspermidine (N1-AcSPD).

(2) The examination method according to the above (1), wherein the polyamines include at least spermine (SPM), N-acetylspermine (N-AcSPM), spermidine (SPD), N1-acetylspermidine (N1-AcSPD), N8-acetylspermidine (N8-AcSPD) and cadaverine (CAD).

(3) The examination method according to the above (1) or (2), wherein the sample collected from a human subject is a saliva sample.

(4) A diagnosis method of breast cancer which comprises analyzing a sample collected from a human subject and determining the presence or absence of breast cancer by a proportion of plural types of polyamines which are selected from a polyamine and a mono- or di-acetyl form thereof and include N1-acetyl spermidine (N1-AcSPD).

(5) The diagnosis method according to the above (4), wherein the polyamines include at least spermine (SPM), N-acetylspermine (N-AcSPM), spermidine (SPD), N1-acetylspermidine (N1-AcSPD), N8-acetylspermidine (N8-AcSPD) and cadaverine (CAD).

(6) The diagnosis method according to the above (4) or (5), wherein the sample collected from a human subject is a saliva sample.

### Effects of the Invention

According to the present invention, it is possible to simply determine breast cancer with high precision.

### BRIEF EXPLANATION OF THE DRAWINGS

FIG. 1 shows the results of principal component analysis. From several hundred to several thousand compounds were detected. From these compounds, markers were searched by OPLS-DA.

FIG. 2 shows the results of S-plot of OPLS-DA analysis.

FIG. 3 shows the hit number of marker candidates. Several ten compounds were picked out as increased or decreased markers. Data base searches were conducted with respect to m/z of respective compounds to inspect the usefulness thereof as the marker.

FIG. 4 shows the results of database search of markers. N-acetyl Spermidine was picked out. Cancer-related report was made concerning N1, N8-diacetyl Spermidine in urine. (ref. J. Cancer Res. Clin Oncol., 123:539-545, 1997)

FIG. 5 shows the structures of polyamines used as the object in measurement.

FIG. 6 shows the outline of a quantitative determination method of polyamines in saliva.

FIG. 7 shows chromatogram and measurement conditions of selected reaction monitoring (SRM) of polyamines.

FIG. 8 shows average values of quantitative determination of polyamines in saliva in each of a healthy person group and a new-onset group of breast cancer patients.

FIG. 9 shows a proportion of average values of quantitative determination of respective polyamines in saliva in each of a healthy person group and a new-onset group of breast cancer patients.

FIG. 10 shows ROC curves of typical four types of polyamines.

FIG. 11 shows the results of ROC analysis. From the polyamines that showed high sensitivity and specificity, several types were chosen for determination analysis. By using plural marker candidates, diagnosis can be made with high precision.

FIG. 12 shows the results obtained by discriminant analysis.

FIG. 13 shows a concordance rate with respect to breast cancer when the examination method of the present invention is used.

### DETAILED DESCRIPTION OF THE INVENTION

As the sample to be used for the present invention, there is no particular limitation so far as the sample is collected from a human subject and contains polyamines selected from a polyamine and its mono- or di-acetyl form. For example, saliva, urine, a blood sample such as blood serum, hair and the like may be mentioned. Among them, saliva is preferred since it can easily be collected and does not bring burdens on patients.

In the present invention, there is no particular limitation in the method for collecting samples from the human subject. For example, in the case of saliva, a method using a collection tube, a sample-collection filter paper, swab or the like may be mentioned.

It is preferred to conduct a pre-treatment of a sample before use depending on each sample. Specifically, it is preferred to conduct a pre-treatment of a saliva sample before use by, for example, dilution with an organic solvent such as acetonitrile, and deproteinization by centrifugation. In usual, a supernatant after deproteinization is used for analysis.

In the present invention, the polyamine is an aliphatic compound having at least two amino groups in one molecule.

The polyamines as the object in the present invention are not particularly limited so far as these are plural types of polyamines including N1-acetylspermidine (N1-AcSPD). As the polyamines other than N1-acetylspermidine (N1-AcSPD), for example, diaminopropane (DAP), putrescine (PUT), N-acetylputrescine (N-AcPUT), cadaverine (CAD), spermidine (SPD), N8-acetylspermidine (N8-AcSPD), diacetyl spermidine (DAcSPD), spermine (SPM), N-acetyl spermine (N-Ac SPM) and diacetylspermine (DAcSPM) may be mentioned.

The polyamines preferably include at least spermine (SPM), N-acetylspermine (N-AcSPM), spermidine (SPD), N1-acetylspermidine (N1-AcSPD), N8-acetylspermidine (N8-AcSPD) and cadaverine (CAD).

Further, the proportion of N1-acetylspermidine (N1-AcSPD) is large in a new-onset group of breast cancer patients and the proportion of putrescine (PUT) is large in a healthy person group, and it is therefore effective to obtain the proportion of polyamines including at least these two types.

As the quantitative determination method of polyamines, there is no particular limitation so far as accurate measurement results can be obtained. For example, LC-MS/MS method and ELISA method may be mentioned, preferably LC-MS/MS (Xevo TQ-S; Waters) method by derivatization using 4-(N,N-dimethylaminosulfonyl)-7-fluoro-2,1,3-benzoxadiazole (DBD-F) as described in Anal. Chem. 2013, 85, 11835-11842 (Non-patent Document 2) may be mentioned.

As the LC-MS/MS (Xevo TQ-S; Waters) method by derivatization using DBD-F, for example, there may be mentioned a method wherein a mixed liquid of an organic solvent (e.g. acetonitrile), a saliva sample and an internal standard (e.g. 1,6-diaminohexane) is centrifuged, thus obtained deproteinized supernatant is subjected to removal of solvent by distillation, the obtained residue is dissolved in an aqueous borax solution or a base such as triethylamine and then subjected to a reaction with 4-(N,N-dimethylaminosulfonyl)-7-fluoro-2,1,3-benzoxadiazole (DBD-F) for derivatization, followed by analysis by UPLC-ESI-MS/MS.

Usually, the obtained analysis results are substituted into a discriminant to conduct diagnosis of breast cancer. The discriminant is not particularly limited so far as it is prepared to ascertain a high concordance rate.

An example of discriminant will be indicated below when six types of polyamines composed of spermine (SPM), N-acetylspermine (N-AcSPM), spermidine (SPD), N1-acetylspermidine (N1-AcSPD), N8-acetylspermidine (N8-AcSPD) and cadaverine (CAD) are used as the polyamines.

Y=AX_{SPM}+BX_{N-AcSPM}+CX_{SPD}+DX_{N8-AcSPD}+EX_{N1-AcSPD}+FX_{CAD}

(Y denotes a discriminant score, X denotes a proportion of respective polyamines when the total of the six polyamines is 100%, A denotes a coefficient “0.5”, B denotes a coefficient “−3”, C denotes a coefficient “−0.15”, D denotes a coefficient “−3.5”, E denotes a coefficient “0.5”, and F denotes a coefficient “0.04”.)

According to the above-mentioned discriminant, a healthy person is recognized when the discriminant score Y is minus, and a new-onset patient of breast cancer is recognized when it is plus.

### EXAMPLES

Hereinafter, the present invention will be described with examples, but it should be mentioned that the present invention is by no means limited to the following examples.

### Example 1

20 Saliva specimens of each of healthy persons and breast cancer patients (new-onset group and recurrence group) were prepared and samples each having 5 saliva specimens pooled out of 20 were prepared, respectively, so as to reduce individual difference, and to thus prepared samples, two-fold amount of acetonitrile was added, and centrifugation was carried out for deproteinization, and then the supernatant was filtrated with a 0.45 μm filter. 10 μL of this supernatant was injected into LC-TOF-MS (Waters) and then analyzed. As the column, a reverse phase (RP) type Discovery™HS F5 (SUPELCO) and a HILIC type TSK-Gel Amide 80 (Tosoh Corporation) were used, and measurements were carried out by ESI positive and negative modes for each column i.e. totally in four systems. The mass range was m/z 100 to 1,000, and the retention time was 0 to 20 minutes in RP and 0 to 25 minutes in HILIC respectively. The obtained chromatogram was analyzed by MarkerLynx (Waters). For the measurement of polyamines, LC-MS/MS (Xevo TQ-S; Waters) method by derivatization using 4-(N,N-dimethylaminosulfonyl)-7-fluoro-2,1,3-benzoxadiazole (DBD-F) as described in Anal. Chem. 2013, 85, 11835-11842 (Non-patent Document 2) was used.

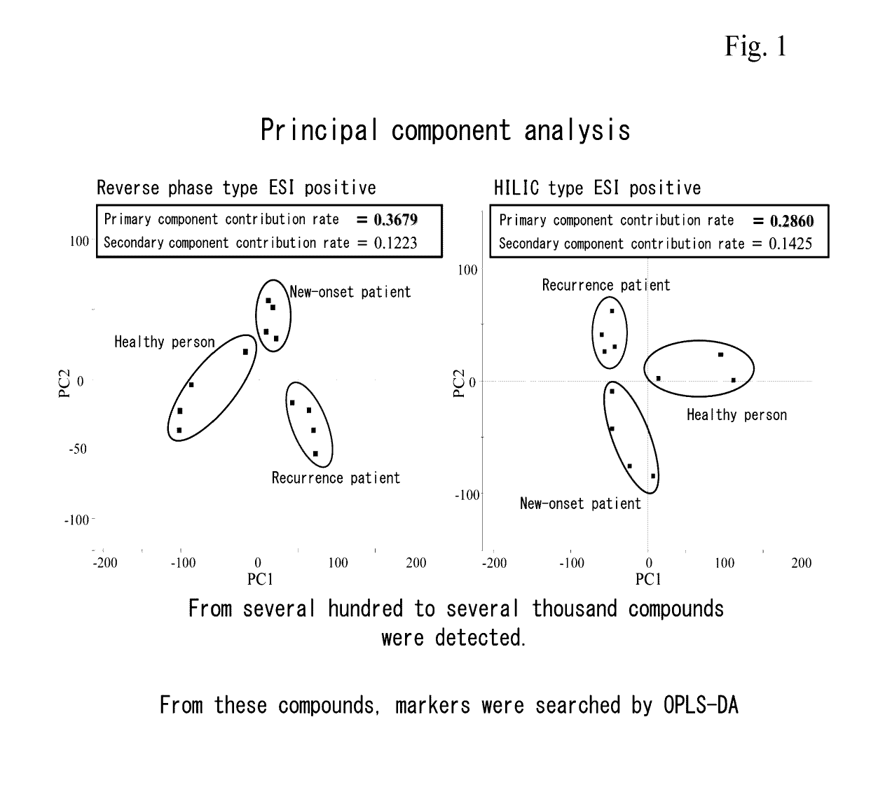

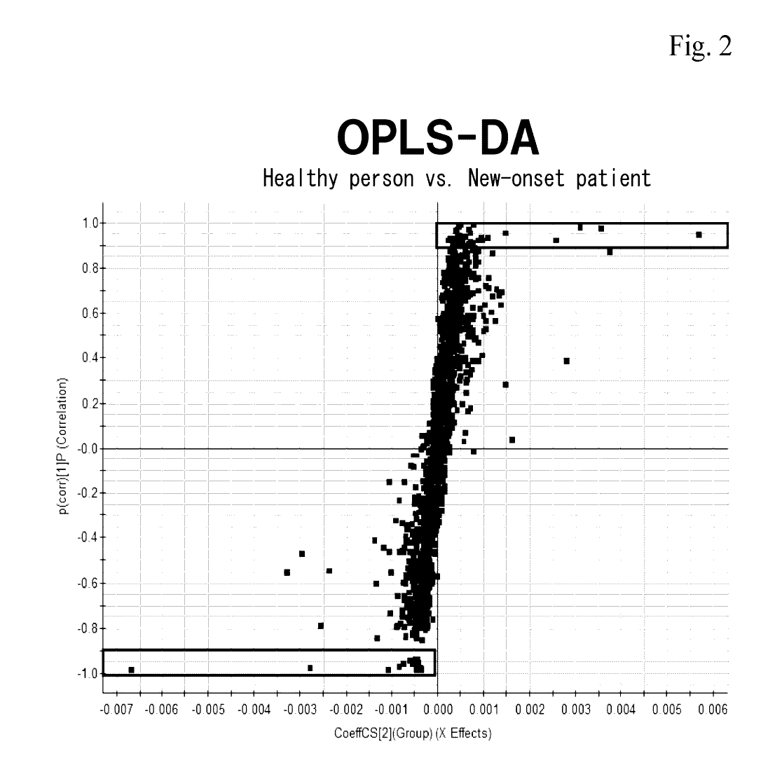

The results of the principal component analysis are shown in FIG. 1. In FIG. 1, one dot shows one sample, and the states of differences in components are reflected in ordinate and abscissa. In all of the measured systems, definite differences were observed among the healthy persons, new-onset group and recurrence group visually and from the viewpoint of contribution rate. Accordingly, it was judged that the components are different among all groups, and OPLS-DA analysis was conducted for the purpose of narrowing down useful markers from detected several hundred to several thousand compounds. The results are shown in FIG. 2.

In the OPLS-DA analysis, group information is further added to the information of the principal component analysis (PCA). The ones indicated in FIG. 2 are S-plots by OPLS-DA analysis, and one dot shows one compound. Ordinate shows the correlation of intensities of detected compounds and means that the dot number increases without dispersion as it reaches close to 1, and decreases without dispersion as it reaches close to −1. Abscissa shows the differences in intensities and means that variation is large as the absolute value increases. Accordingly, it can be judged that useful markers are present at the right upper area and the left lower area. In this example, the markers having an absolute value of at least 0.9 in the ordinate were tested in detail.

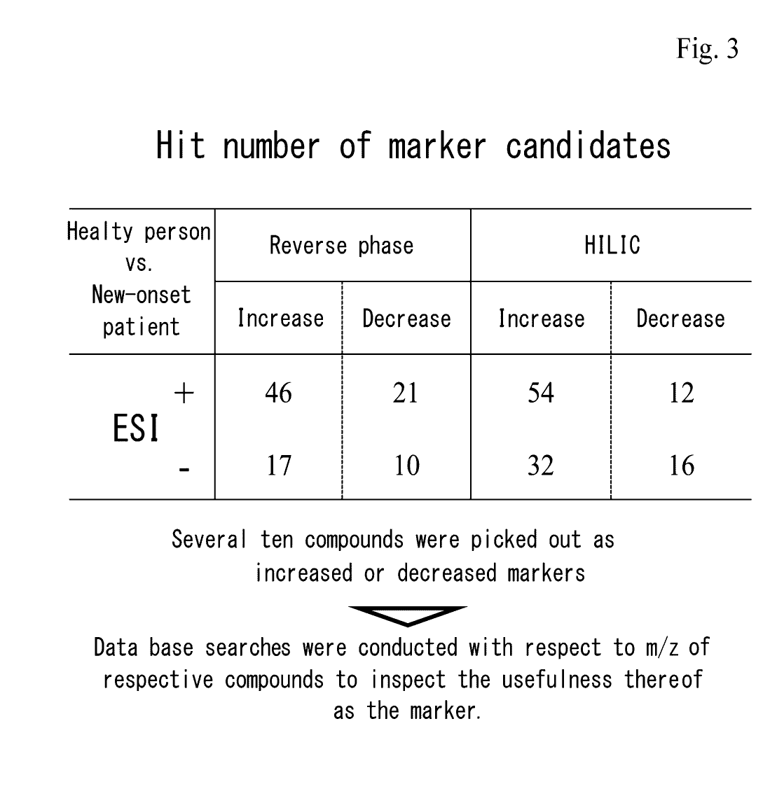

The plots indicated in FIG. 2 were drawn for every system, and the numbers of compounds included in the black-framed areas at the left lower side and the right upper side of FIG. 2 are indicated in FIG. 3. In every system, about double-figure numbers of compounds were picked out. In the present invention, it was particularly expected to find out markers distinctive between healthy persons and new-onset patients, and therefore searches were made aiming to find them.

As the results of database searches, HILIC type ESI positive mode results from which particularly effective information was obtained are indicated as one example.

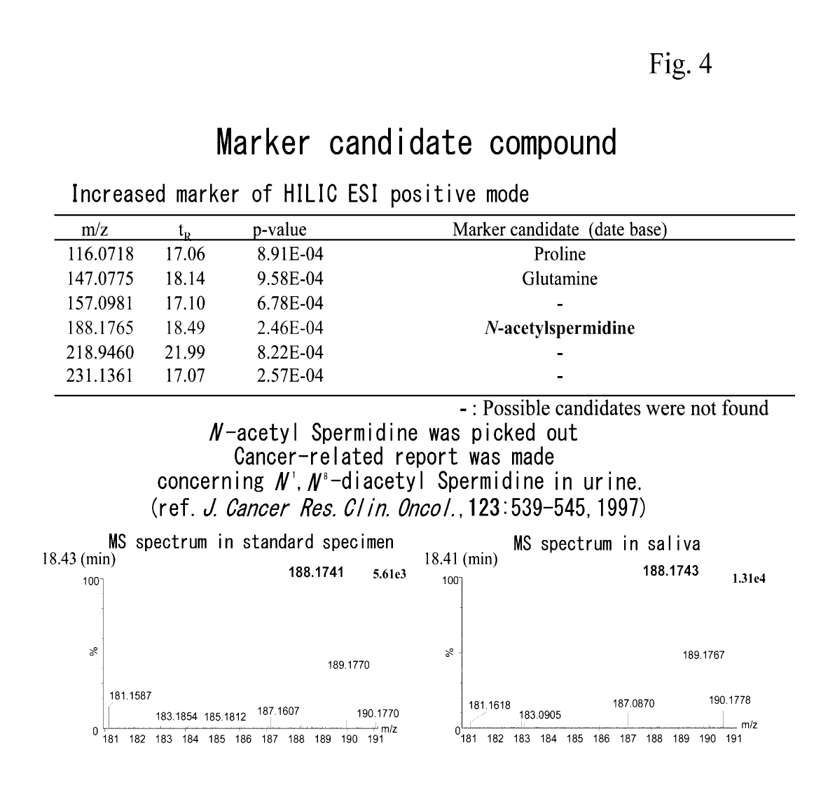

FIG. 4 shows the database search results of makers which were judged to probably have high usefulness in view of peak shapes, intensities, etc. among the picked-out 54 types of compounds that showed increase. It is considered that N1-acetylspermidine is particularly useful since cancer-related reports have been made in that N1-acetylspermidine is increased in blood serum and urine. Accordingly, a standard specimen was prepared and tests for identification were conducted by confirming the similarity of the retention time and MS behavior.

As the results, as indicated in the spectrum of FIG. 4, the data of the saliva sample agreed with the standard specimen, and therefore it was identified as N1-acetylspermidine.

Then, it was also considered that further information may be obtained by quantitative determination with respect to increase or decrease of other polyamines, and simultaneous analysis including them was tried.

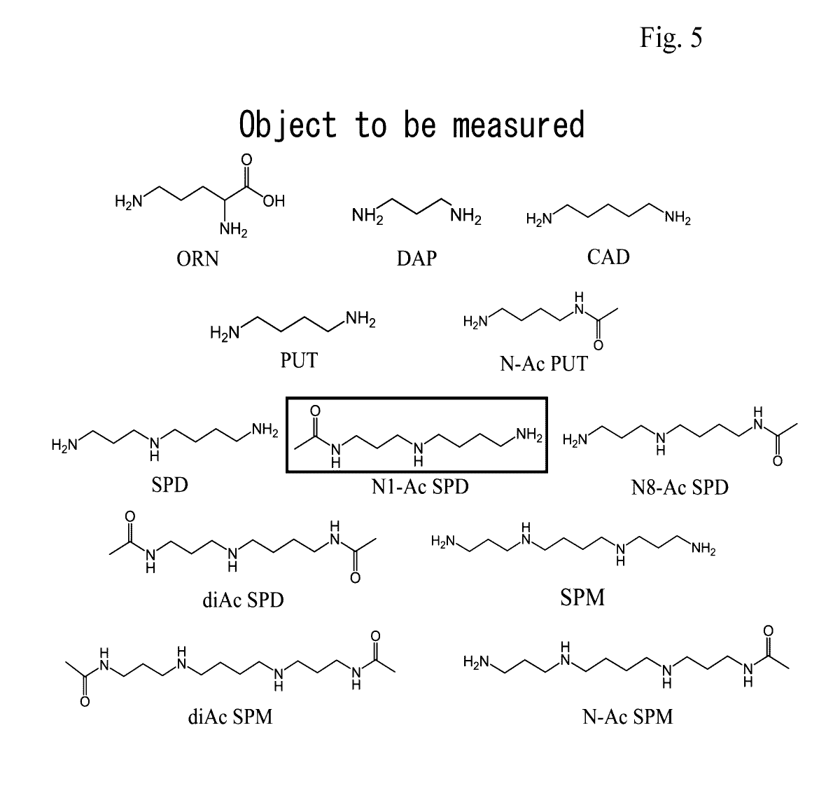

As the marker candidates, in addition to the picked-out N1-acetylspermidine (N1-AcSPD), 11 types of polyamines were prepared. The structures of these polyamines are indicated in FIG. 5. Further, although not shown in FIG. 5, 1,6-diaminohexane (DAH) was used as an internal standard (IS).

The method used for the quantitative determination of polyamines in saliva was LC-MS/MS (Xevo TQ-S; Waters) method by derivatization using 4-(N,N-dimethylaminosulfonyl)-7-fluoro-2,1,3-benzoxadiazole (DBD-F) as described in Anal. Chem. 2013, 85, 11835-11842 (Non-patent Document 2).

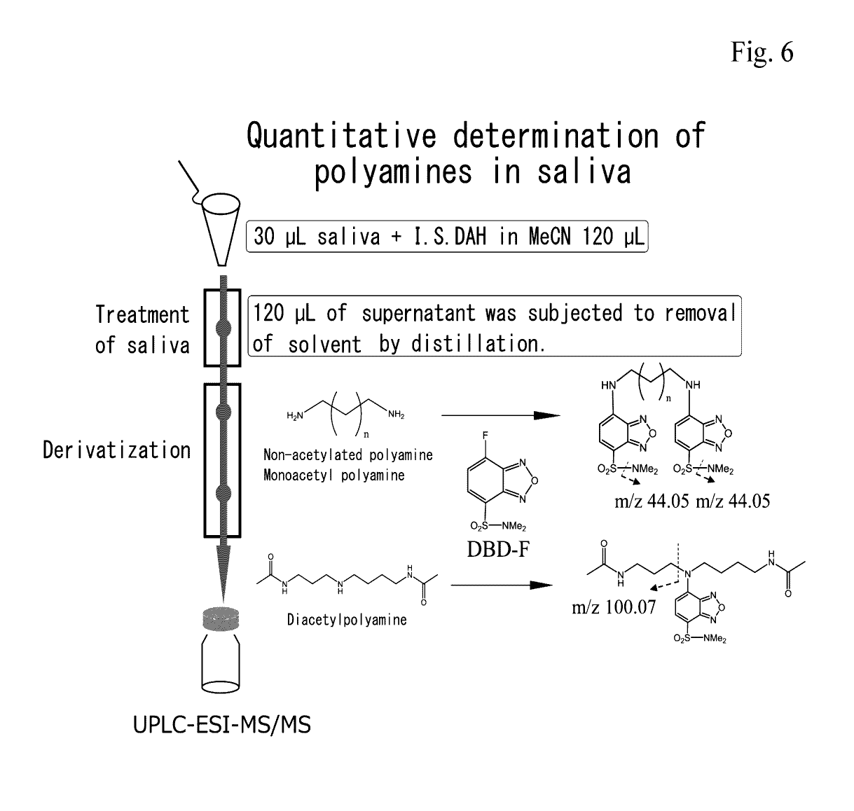

Outline of the quantitative determination method of polyamines in saliva is indicated in FIG. 6.

A mixed liquid of 30 μL of saliva sample and 1,6-diaminohexane (DAH) as an internal standard (IS) in 120 μL of acetonitrile was subjected to centrifugation at 3,000 g for 10 minutes for deproteinization, and 120 μL of thus obtained supernatant was subjected to removal of solvent by distillation. The thus obtained residue was dissolved in a 0.1M borax aqueous solution or a base such as triethylamine and then subjected to a reaction with 4-(N,N-dimethylaminosulfonyl)-7-fluoro-2,1,3-benzoxadiazole (DBD-F) for derivatization, and then subjected to analysis by UPLC-ESI-MS/MS.

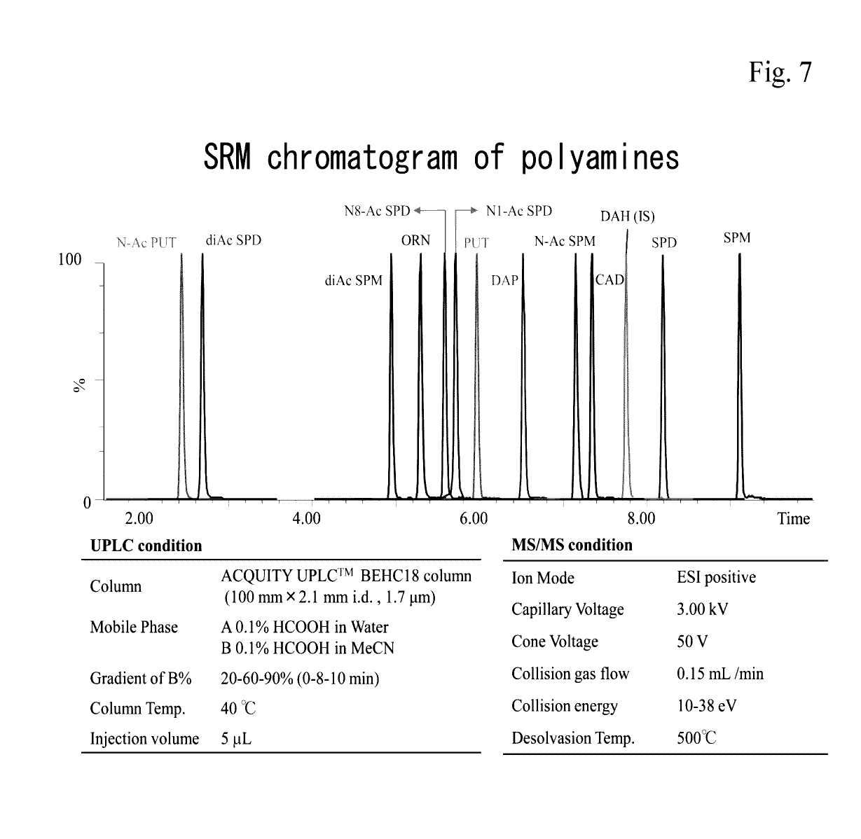

FIG. 7 shows the chromatogram of the measurement results and the measurement conditions. As the column, a reverse phase type ODS column was used. The measurement was conducted under the mobile phase conditions as shown in FIG. 7, and all of 13 types of polyamines including 1,6-diaminohexane (DAH) as internal standard (IS) were separated and detected within 10 minutes.

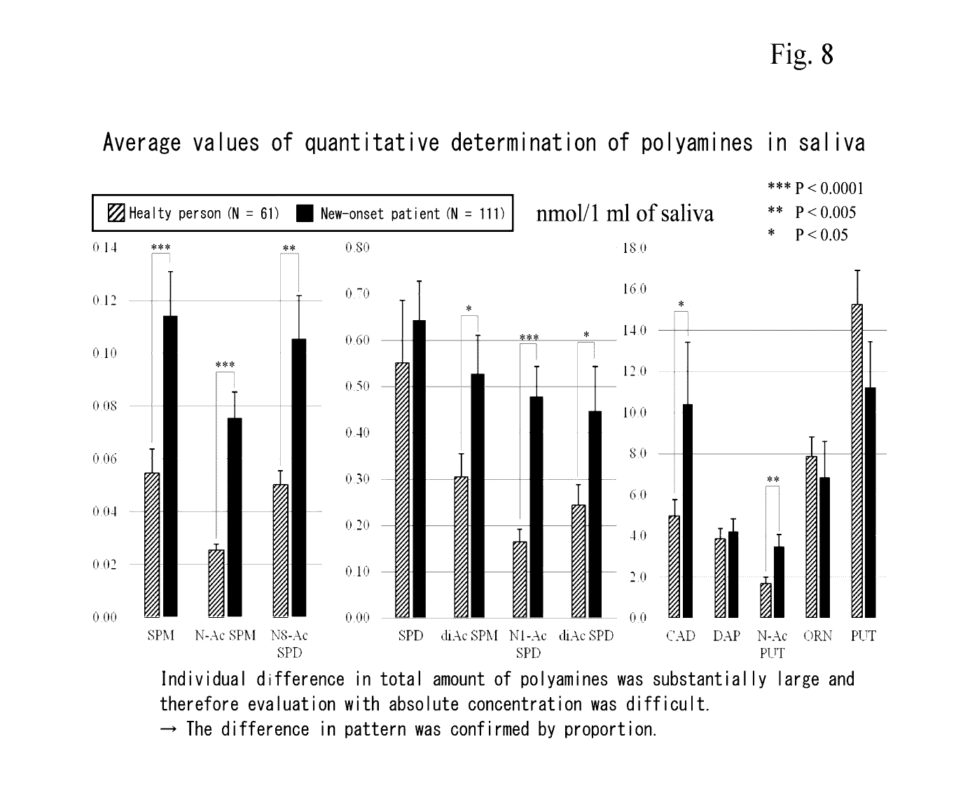

FIG. 8 shows graphs of average values of quantitative determination of each of the healthy person group (61 examples) and the breast cancer new-onset patient group (111 examples). The concentrations were adjusted in nmol/1 ml of saliva.

Almost all of the polyamines showed the tendency in that the measured values were high in the patient group, and it is self-evident that significant difference is observed by t-test particularly in spermine type and spermidine type metabolites.

However, as the results of detailed studies, the individual difference in total amount was substantially large and therefore it was considered that erroneous determination may be caused when the cutoff value is set on the basis of absolute concentration. Accordingly, in order to minimize the influence of individual difference in total amount, it was tried to compare the difference in pattern by proportion.

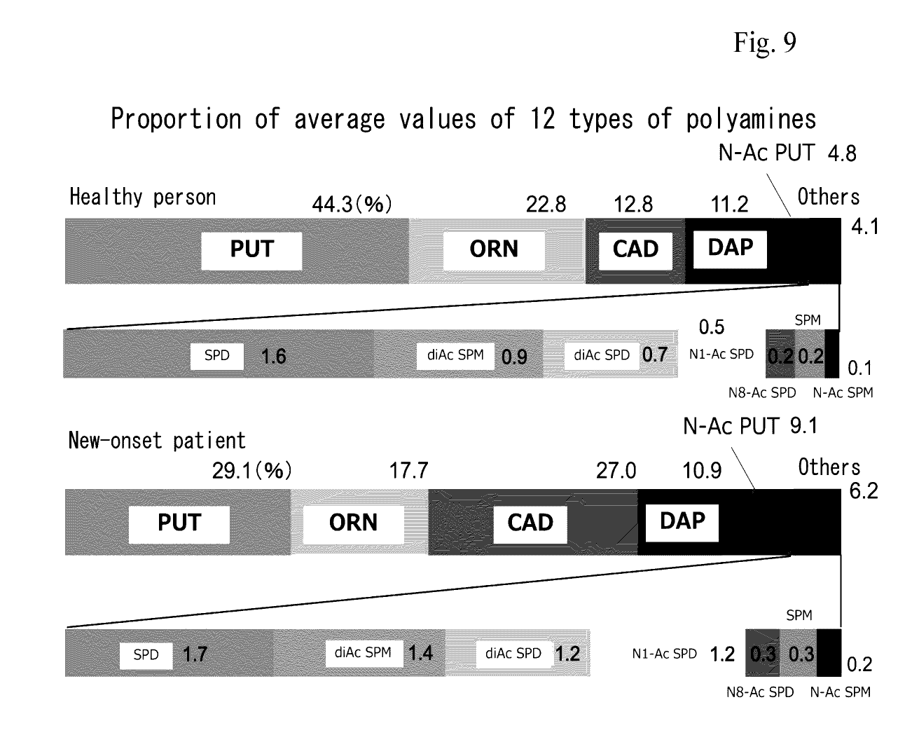

FIG. 9 shows the proportion of average values measured by quantitative determination of respective polyamines in saliva in each of the healthy person group in the upper section and the breast cancer new-onset patient group in the lower section. From this figure, difference can be seen visually.

For example, the proportion of N1-acetylspermidine (N1-AcSPD) is large in the new-onset group of breast cancer patients and the proportion of putrescine (PUT) is large in the healthy person group.

Accordingly, the values of proportion were used for ROC analysis and a cutoff value was set as a diagnostic index.

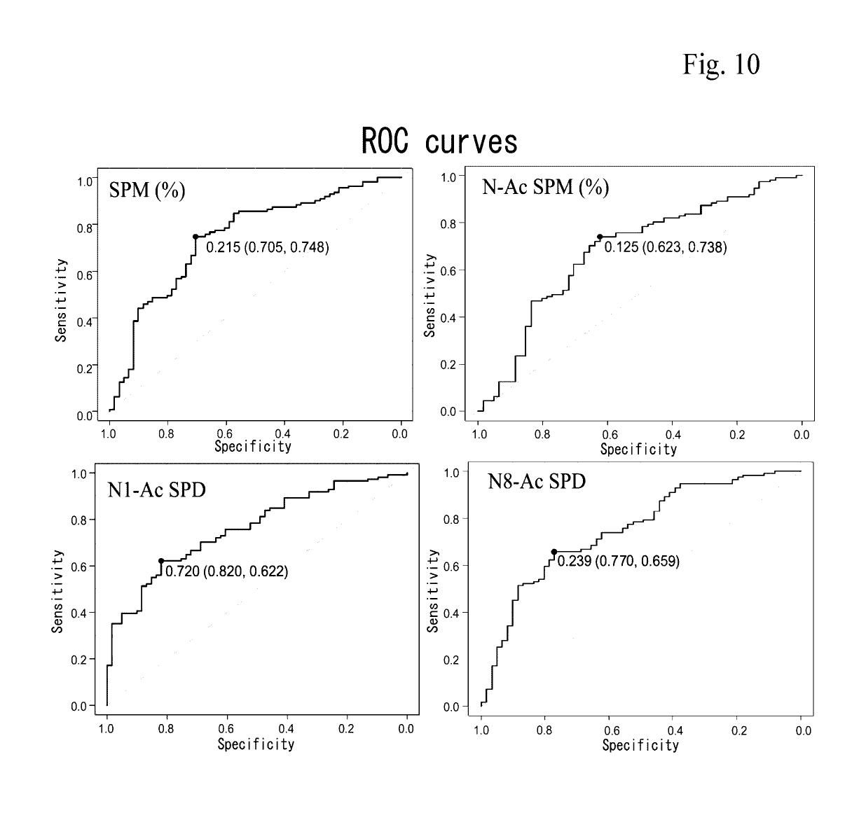

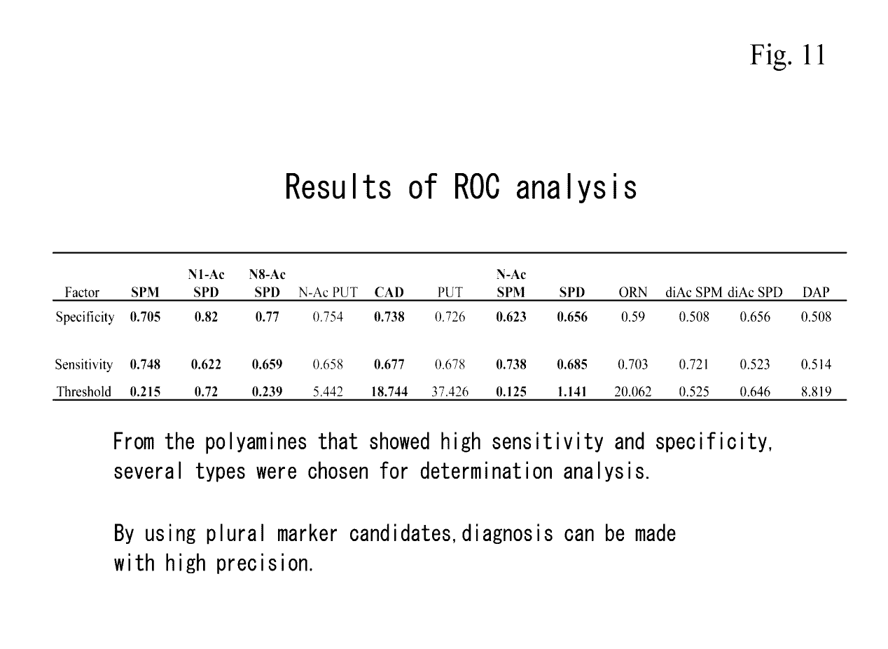

FIGS. 10 and 11 show the results of ROC analysis. The four curves shown in FIG. 10 are typical examples. Further, FIG. 11 shows the results of ROC analysis of 12 types of polyamines.

Although it seemed that enough difference was observed visually, the scores of these polyamines were around 65% and at such a level, these were not reliable as markers.

Taking the results into consideration, discriminant analysis was limited to the ones of which the sum of the sensitivity and specificity was particularly large, to find out a diagnosis method with a high precision.

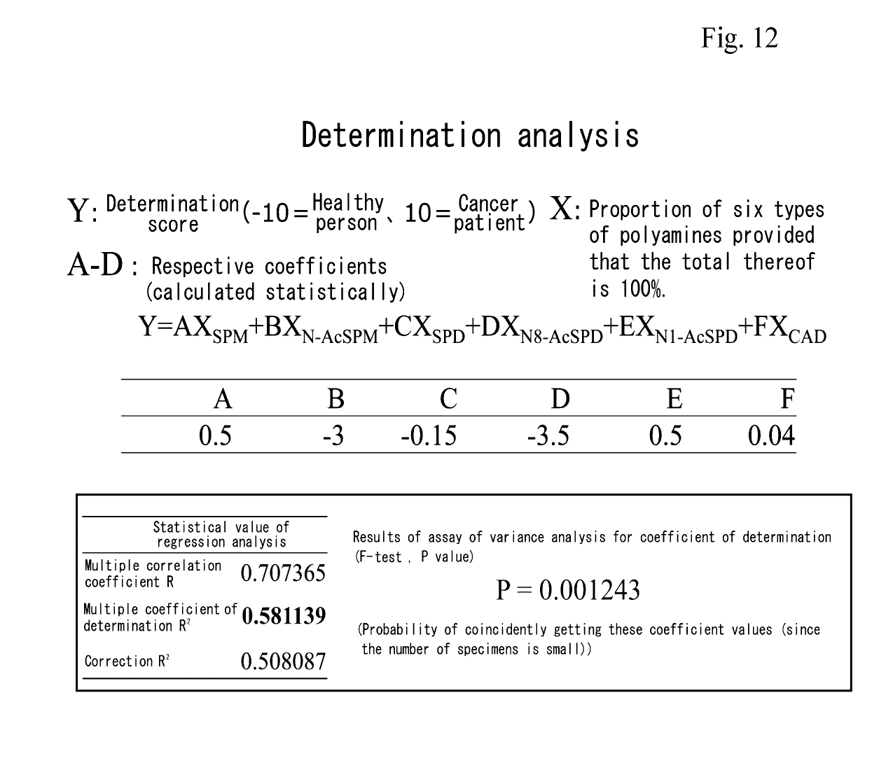

FIG. 12 shows the results obtained by the discriminant analysis.

The discriminant was statistically optimized, and the linear equation with multiple unknowns as indicated was obtained with respective specific coefficients. As the calculation conditions, as indicated in FIG. 12, healthy person was given −10 points and new-onset patient was given 10 points as Y Accordingly, if Y is negative, a composition of polyamines toward healthy person side is obtained from the saliva, and if Y is positive, a composition of polyamines toward a new-onset patient side is obtained from the saliva.

It was tried to confirm by statistical value of regression analysis whether the discriminant is a model close to the population, and as the results, its multiple correlation coefficient was at least 0.5 and the P value of variance analysis related thereto was around 0.1%, and therefore the results showed that enough usefulness can be obtained.

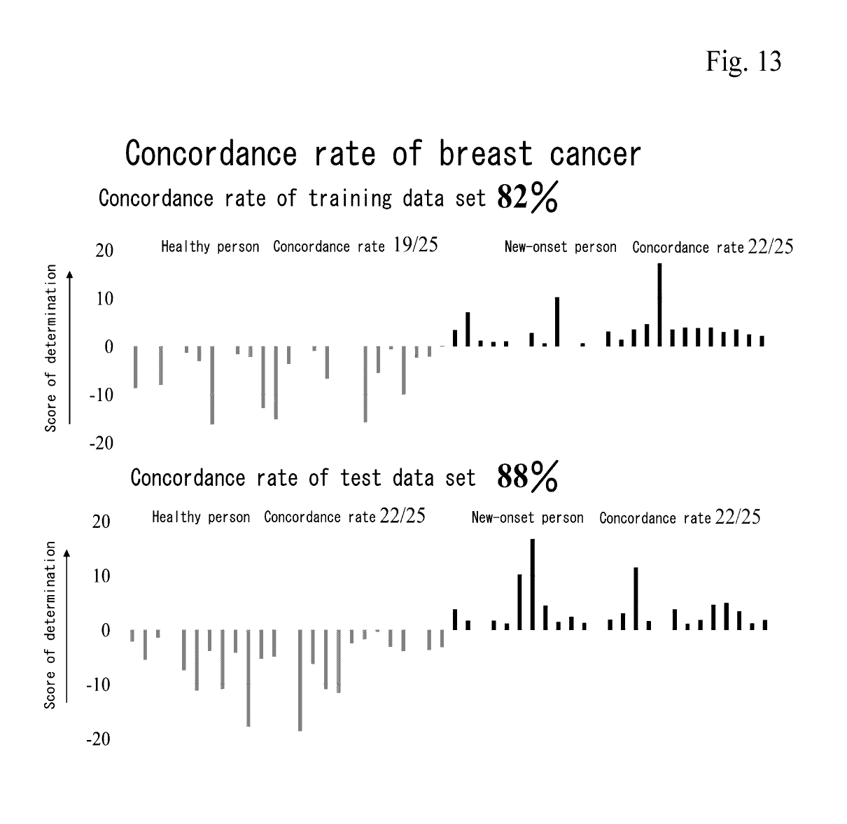

The prepared set was substituted into the discriminant indicated in FIG. 12 and the concordance rate was 82% in the entire samples, and other specimen set was substituted and the result was 88%, namely the concordance rate of about 85% was obtained (FIG. 13).

From the above, it can be concluded that the examination process of the present invention is applicable to diagnosis of breast cancer using a biological specimen.

1. An examination method of breast cancer which comprises analyzing a sample collected from a human subject and determining the presence or absence of breast cancer by a proportion of plural types of polyamines which are selected from a polyamine and a mono- or di-acetyl form thereof and include N1-acetylspermidine.

2. The examination method according to claim 1, wherein the polyamines include at least spermine, N-acetylspermine, spermidine, N1-acetylspermidine, N8-acetylspermidine and cadaverine (CAD).

3. The examination method according to claim 1, wherein the sample collected from a human subject is a saliva sample.

4. The examination method according to claim 2, wherein the sample collected from a human subject is a saliva sample.

5. A diagnosis method of breast cancer which comprises analyzing a sample collected from a human subject and determining the presence or absence of breast cancer by a proportion of plural types of polyamines which are selected from a polyamine and a mono- or di-acetyl form thereof and include N1-acetylspermidine.

6. The diagnosis method according to claim 5, wherein the polyamines include at least spermine, N-acetylspermine, spermidine, N1-acetylspermidine, N8-acetylspermidine and cadaverine (CAD).

7. The diagnosis method according to claim 5, wherein the sample collected from a human subject is a saliva sample.

8. The diagnosis method according to claim 6, wherein the sample collected from a human subject is a saliva sample.
